(19) United States
(12) Patent Application Publication
(10) Pub. No.: US 2026/0086272 A1
(43) Pub. Date: Mar. 26, 2026

(54) ANNULAR SHAPED PHOSPHOR IN COMBINATION WITH AXICON LENS FOR PRODUCING LASER PUMPED HIGH INTENSITY WHITE LIGHT SOURCE

(71) Applicant: SIGNIFY HOLDING B.V., EINDHOVEN (NL)

(72) Inventors: RIFAT ATA MUSTAFA HIKMET, EINDHOVEN (NL); HUGO JOHAN CORNELISSEN, ESCHAREN (NL); OLEXANDR VALENTYNOVYCH VDOVIN, HORST (NL); TIES VAN BOMMEL, HORST (NL)

(21) Appl. No.: 19/109,752

(22) PCT Filed: Aug. 31, 2023
(86) PCT No.: PCT/EP2023/073869

§ 371 Date: Mar. 7, 2025

(30) Foreign Application Priority Data
Sep. 9, 2022 (EP) 22194760.9

(51) Int. Cl. G02B 5/00; A61L 2/08; F21V 5/04; F21V 9/32; G03B 21/20

(52) U.S. Cl. CPC G02B 5/001; A61L 2/084; F21V 5/04; F21V 9/32; G03B 21/204; A61L 2202/11

(57) ABSTRACT

The invention provides a light generating system (1000) comprising a first light generating device (110), a luminescent body (1200), a thermally conductive element (500), and an axicon-like optical element (400); wherein: (A) the first light generating device (110) is configured to generate first device light (111); the first light generating device (110) comprises one or more of a superluminescent diode and a solid state laser; (B) the luminescent body (1200) comprises a luminescent material (200) configured to convert at least part of the first device light (111) into luminescent material light (201); the luminescent body (1200) has an annular shape; (C) the thermally conductive element (500) (a) is configured in thermal contact with at least part of the luminescent body (1200), and (b) is reflective for one or more of the first device light (111) and the luminescent material light (201); (D) the axicon-like optical element (400) comprises a first part (410) and a second part (420), and has an optical element length (L); the first part (410) has a conical shape, a first length (L1), and comprises a first end window (411); the second part (420) has a cylindrical shape, a second length (L2), and comprises a second end window (422); wherein 0.7≤L2/L<1; and (E) the axicon-like optical element (400) is configured to: (a) receive at least part of the first device light (111) via the first part (410) and provide an annular beam of first device light (111) via the second part (420) to the luminescent body (1200), and (b) collect at least part of the luminescent material light (201) via the second part (420) and provide a beam of luminescent material light (201) via the first part (410).

### FIELD OF THE INVENTION

The invention relates to a light generating system and to a light generating device comprising such light generating system.

### BACKGROUND OF THE INVENTION

Solid state lighting is known in the art. For instance, U.S. Pat. No. 7,165,871 B2 describes a lamp for generating light which comprises a semiconductor light emitting element for emitting light, a fluorescent material, provided away from the semiconductor light emitting element, a first optical member operable to focus the light generated by the semiconductor light emitting element on the fluorescent material, and a second optical member having an optical center at a position where the fluorescent material is provided, operable to emit light from the fluorescent material based on the light focused by the optical member to an outside of the lamp. The lamp is used as a headlamp in a vehicle, and the second optical member emits the light from the fluorescent material to the outside of the lamp, so that the second optical member forms at least one of a part of a cut line that defines a boundary between a bright region and a dark region of the headlamp. This document further describes the combination of a laser, a phosphor and a reflector integrated into a light emitting module used for automotive front light applications.

### SUMMARY OF THE INVENTION

Laser based light sources are gathering much interest due to their potential in producing relatively high flux from relatively small light emitting areas. The high brightness of these sources may facilitate miniaturization and more precise control of light distribution with optics. It may further be desired to have a high brightness light source for general lighting applications tunable in the broad range of color space/CCTs with good color rendering. Usually, to achieve color tuneability, a combination of several sources with different starting color points may be required (being e.g. various sources with different phosphors, different primary colors from direct emitters (e.g. RGB) or a combination of those). In order to create a high brightness color-tunable light source, these multiple sources may need to be optically combined with good color mixing, and without additional increase of etendue. However, for systems with direct RGB lasers, barring impractical primary laser wavelengths requirements, e.g. due to intrinsic narrow spectral width of laser lines and/or practical limitations, e.g. to certain limited spectral ranges, the optical combination of multiple sources often results in a relatively low CRI. Further, for systems with more than one phosphor converter, the etendue tends to increase substantially (such as at least ×2 times), which may be undesired for high brightness applications. Further, prior art systems may require a multi-channel driver and/or additional color mixing. Further, it may be desirable to use commonly available light sources, rather than requiring specialty equipment.

It appears that there is a desire for lighting devices which may be relatively compact and/or provide a relatively high intensity. Further, it appears desirable that the heat management of such lighting devices is sufficient, such that such high light intensities may be possible by using high intensity pump light sources, such as e.g. lasers.

Hence, it is an aspect of the invention to provide an alternative light generating system (and/or lighting device (comprising such light generating system)), which preferably further at least partly obviates one or more of above-described drawbacks. The present invention may have as object to overcome or ameliorate at least one of the disadvantages of the prior art, or to provide a useful alternative.

According to a first aspect, the invention provides a light generating system (“system”) comprising a first light generating device, a luminescent body, a thermally conductive element, and an axicon-like optical element. Especially, the first light generating device is configured to generate first device light. In specific embodiments, the first light generating device may comprise one or more of a superluminescent diode and a solid state laser. Further, especially the luminescent body comprises a luminescent material which may be configured to convert at least part of the first device light into luminescent material light. In specific embodiments, the luminescent body may have an annular shape. In embodiments, the thermally conductive element may be configured in thermal contact with at least part of the luminescent body. In specific embodiments, the thermally conductive body may be reflective for one or more of the first device light and the luminescent material light. In specific embodiments, the axicon-like optical element may comprise a first part and a second part, and may have an optical element length (L). In further specific embodiments, the first part may have a conical shape, may have a first length (L1), and may comprise a first end window. Yet, in further specific embodiments the second part may have a cylindrical shape, may have a second length (L2), and may comprise a second end window. Especially, in embodiments 0.7≤L2/L<1 may apply. In specific embodiments, the axicon-like optical element may be configured to receive at least part of the first device light via the first part and provide an annular beam of first device light via the second part to the luminescent body. In specific embodiments, the axicon-like optical element may also be configured to collect at least part of the luminescent material light via the second part and provide a beam of luminescent material light via the first part. Therefore, the invention especially provides a light generating system comprising a first light generating device, a luminescent body, a thermally conductive element, and an axicon-like optical element; wherein: (A) the first light generating device is configured to generate first device light; the first light generating device comprises one or more of a superluminescent diode and a solid state laser; (B) the luminescent body comprises a luminescent material configured to convert at least part of the first device light into luminescent material light; the luminescent body has an annular shape; (C) the thermally conductive element (a) is configured in thermal contact with at least part of the luminescent body, and (b) is reflective for one or more of the first device light and the luminescent material light; (D) the axicon-like optical element comprises a first part and a second part, and has an optical element length (L); the first part has a conical shape, a first length (L1), and comprises a first end window; the second part has a cylindrical shape, a second length (L2), and comprises a second end window; wherein 0.7≤L2/L<1; and (E) the axicon-like optical element is configured to: (a) receive at least part of the first device light via the first part and provide an annular beam of first device light via the second part to the luminescent body, and (b) collect at least part of the luminescent material light via the second part and provide a beam of luminescent material light via the first part.

With such system, it may be possible to provide light with a relatively high intensity. Further, with such system it may be possible to use a high intensity light source to pump the luminescent material. Yet, such system may be relatively compact. In embodiments, it may also be possible to provide white (system) light with such system.

As indicated above, the invention provides a light generating system comprising a first light generating device, a luminescent body, a thermally conductive element, and an axicon-like optical element. These will be described below.

A light generating device may especially be configured to generate device light. Especially, the light generating device may comprise a light source. The light source may especially configured to generate light source light. In embodiments, the device light may essentially consist of the light source light. In other embodiments, the device light may essentially consist of converted light source light. In yet other embodiments, the device light may comprise (unconverted) light source light and converted light source light. Light source light may be converted with a luminescent material into luminescent material light and/or with an upconverter into upconverted light (see also below). The term “light generating device” may also refer to a plurality of light generating devices which may provide device light having essentially the same spectral power distributions. In specific embodiments, the term “light generating device” may also refer to a plurality of light generating devices which may provide device light having different spectral power distributions.

The term “light source” may in principle relate to any light source known in the art. It may be a conventional (tungsten) light bulb, a low pressure mercury lamp, a high pressure mercury lamp, a fluorescent lamp, an LED (light emissive diode). In a specific embodiment, the light source comprises a solid state LED light source (such as an LED or laser diode (or “diode laser”)). The term “light source” may also relate to a plurality of light sources, such as 2-2000 (solid state) LED light sources. Hence, the term LED may also refer to a plurality of LEDs. Further, the term “light source” may in embodiments also refer to a so-called chip-on-board (COB) light source. The term “COB” especially refers to LED chips in the form of a semiconductor chip that is neither encased nor connected but directly mounted onto a substrate, such as a PCB. Hence, a plurality of light emitting semiconductor light source may be configured on the same substrate. In embodiments, a COB is a multi LED chip configured together as a single lighting module.

The term “light source” may also refer to a chip scaled package (CSP). A CSP may comprise a single solid state die with provided thereon a luminescent material comprising layer. The term “light source” may also refer to a midpower package. A midpower package may comprise one or more solid state die(s). The die(s) may be covered by a luminescent material comprising layer. The die dimensions may be equal to or smaller than 2 mm, such as in the range of e.g. 0.2-2 mm. Hence, in embodiments the light source comprises a solid state light source. Further, in specific embodiments, the light source comprises a chip scale packaged LED. Herein, the term “light source” may also especially refer to a small solid state light source, such as having a mini size or micro size. For instance, the light sources may comprise one or more of mini LEDs and micro LEDs. Especially, in embodiment the light sources comprise micro LEDs or “microLEDs” or “μLEDs”. Herein, the term mini size or mini LED especially indicates to solid state light sources having dimensions, such as die dimension, especially length and width, selected from the range of 100 μm-1 mm. Herein, the term u size or micro LED especially indicates to solid state light sources having dimensions, such as die dimension, especially length and width, selected from the range of 100 μm and smaller.

The light source may have a light escape surface. Referring to conventional light sources such as light bulbs or fluorescent lamps, it may be an outer surface of a glass or a quartz envelope. For LED's it may for instance be the LED die, or when a resin is applied to the LED die, the outer surface of the resin. In principle, it may also be the terminal end of a fiber. The term escape surface especially relates to that part of the light source, where the light actually leaves or escapes from the light source. The light source is configured to provide a beam of light. This beam of light (thus) escapes from the light exit surface of the light source.

Likewise, a light generating device may comprise a light escape surface, such as an end window. Further, likewise a light generating system may comprise a light escape surface, such as an end window.

The term “light source” may refer to a semiconductor light-emitting device, such as a light emitting diode (LEDs), a resonant cavity light emitting diode (RCLED), a vertical cavity laser diode (VCSELs), an edge emitting laser, etc. The term “light source” may also refer to an organic light-emitting diode (OLED), such as a passive-matrix (PMOLED) or an active-matrix (AMOLED). In a specific embodiment, the light source comprises a solid-state light source (such as an LED or laser diode). In an embodiment, the light source comprises an LED (light emitting diode). The terms “light source” or “solid state light source” may also refer to a superluminescent diode (SLED).

The term LED may also refer to a plurality of LEDs.

The term “light source” may also relate to a plurality of (essentially identical (or different)) light sources, such as 2-2000 solid state light sources. In embodiments, the light source may comprise one or more micro-optical elements (array of micro lenses) downstream of a single solid-state light source, such as an LED, or downstream of a plurality of solid-state light sources (i.e. e.g. shared by multiple LEDs). In embodiments, the light source may comprise an LED with on-chip optics. In embodiments, the light source comprises pixelated single LEDs (with or without optics) (offering in embodiments on-chip beam steering).

In embodiments, the light source may be configured to provide primary radiation, which is used as such, such as e.g. a blue light source, like a blue LED, or a green light source, such as a green LED, and a red light source, such as a red LED. Such LEDs, which may not comprise a luminescent material (“phosphor”) may be indicated as direct color LEDs.

In other embodiments, however, the light source may be configured to provide primary radiation and part of the primary radiation is converted into secondary radiation. Secondary radiation may be based on conversion by a luminescent material. The secondary radiation may therefore also be indicated as luminescent material radiation. The luminescent material may in embodiments be comprised by the light source, such as an LED with a luminescent material layer or dome comprising luminescent material. Such LEDs may be indicated as phosphor converted LEDs or PC LEDs (phosphor converted LEDs). In other embodiments, the luminescent material may be configured at some distance (“remote”) from the light source, such as an LED with a luminescent material layer not in physical contact with a die of the LED. Hence, in specific embodiments the light source may be a light source that during operation emits at least light at wavelength selected from the range of 380-470 nm. However, other wavelengths may also be possible. This light may partially be converted by the luminescent material.

In embodiments, the light generating device may comprise a luminescent material. In embodiments, the light generating device may comprise a PC LED. In other embodiments, the light generating device may comprise a direct LED (i.e. no phosphor). In embodiments, the light generating device may comprise a laser device, like a laser diode. In embodiments, the light generating device may comprise a superluminescent diode. Hence, in specific embodiments, the light source may be selected from the group of laser diodes and superluminescent diodes. In other embodiments, the light source may comprise an LED.

The light source may especially be configured to generate light source light having an optical axis (O), (a beam shape,) and a spectral power distribution. The light source light may in embodiments comprise one or more bands, having band widths as known for lasers.

The term “light source” may (thus) refer to a light generating element as such, like e.g. a solid state light source, or e.g. to a package of the light generating element, such as a solid state light source, and one or more of a luminescent material comprising element and (other) optics, like a lens, a collimator. A light converter element (“converter element” or “converter”) may comprise a luminescent material comprising element. For instance, a solid state light source as such, like a blue LED, is a light source. A combination of a solid state light source (as light generating element) and a light converter element, such as a blue LED and a light converter element, optically coupled to the solid state light source, may also be a light source (but may also be indicated as light generating device). Hence, a white LED is a light source (but may e.g. also be indicated as (white) light generating device).

The term “light source” herein may also refer to a light source comprising a solid state light source, such as an LED or a laser diode or a superluminescent diode.

The term “light source” may (thus) in embodiments also refer to a light source that is (also) based on conversion of light, such as a light source in combination with a luminescent converter material. Hence, the term “light source” may also refer to a combination of an LED with a luminescent material configured to convert at least part of the LED radiation, or to a combination of a (diode) laser with a luminescent material configured to convert at least part of the (diode) laser radiation.

In embodiments, the term “light source” may also refer to a combination of a light source, like an LED, and an optical filter, which may change the spectral power distribution of the light generated by the light source. Especially, the term “light generating device” may be used to address a light source and further (optical components), like an optical filter and/or a beam shaping element, etc.

The phrases “different light sources” or “a plurality of different light sources”, and similar phrases, may in embodiments refer to a plurality of solid-state light sources selected from at least two different bins. Likewise, the phrases “identical light sources” or “a plurality of same light sources”, and similar phrases, may in embodiments refer to a plurality of solid-state light sources selected from the same bin.

The term “solid state light source”, or “solid state material light source”, and similar terms, may especially refer to semiconductor light sources, such as a light emitting diode (LED), a diode laser, or a superluminescent diode.

The term “laser light source” especially refers to a laser. Such laser may especially be configured to generate laser light source light having one or more wavelengths in the UV, visible, or infrared, especially having a wavelength selected from the spectral wavelength range of 200-2000 nm, such as 300-1500 nm. The term “laser” especially refers to a device that emits light through a process of optical amplification based on the stimulated emission of electromagnetic radiation.

Especially, in embodiments the term “laser” may refer to a solid-state laser. In specific embodiments, the terms “laser” or “laser light source”, or similar terms, refer to a laser diode (or diode laser).

Hence, in embodiments the light source comprises a laser light source. In embodiments, the terms “laser” or “solid state laser” or “solid state material laser” may refer to one or more of cerium doped lithium strontium (or calcium) aluminum fluoride (Ce:LiSAF, Ce:LiCAF), chromium doped chrysoberyl (alexandrite) laser, chromium ZnSe (Cr:ZnSe) laser, divalent samarium doped calcium fluoride (Sm:CaF₂) laser, Er:YAG laser, erbium doped and erbium-ytterbium codoped glass lasers, F-Center laser, holmium YAG (Ho:YAG) laser, Nd:YAG laser, NdCrYAG laser, neodymium doped yttrium calcium oxoborate Nd:YCa₄O(BO₃)₃ or Nd:YCOB, neodymium doped yttrium orthovanadate (Nd:YVO₄) laser, neodymium glass (Nd:glass) laser, neodymium YLF (Nd:YLF) solid-state laser, promethium 147 doped phosphate glass (147Pm³⁺:glass) solid-state laser, ruby laser (Al₂O₃:Cr³⁺), thulium YAG (Tm:YAG) laser, titanium sapphire (Ti:sapphire; Al₂O₃:Ti³⁺) laser, trivalent uranium doped calcium fluoride (U:CaF₂) solid-state laser, Ytterbium doped glass laser (rod, plate/chip, and fiber), Ytterbium YAG (Yb:YAG) laser, Yb₂O₃ (glass or ceramics) laser, etc.

For instance, including second and third harmonic generation embodiments, the light source may comprise one or more of an F center laser, an yttrium orthovanadate (Nd:YVO₄) laser, a promethium 147 doped phosphate glass (147Pm³⁺:glass), and a titanium sapphire (Ti:sapphire; Al₂O₃:Ti³⁺) laser. For instance, considering second and third harmonic generation, such light sources may be used to generated blue light.

In embodiments, the terms “laser” or “solid state laser” or “solid state material laser” may refer to one or more of a semiconductor laser diodes, such as GaN, InGaN, AlGaInP, AlGaAs, InGaAsP, lead salt, vertical cavity surface emitting laser (VCSEL), quantum cascade laser, hybrid silicon laser, etc.

A laser may be combined with an upconverter in order to arrive at shorter (laser) wavelengths. For instance, with some (trivalent) rare earth ions upconversion may be obtained or with non-linear crystals upconversion can be obtained. Alternatively, a laser can be combined with a downconverter, such as a dye laser, to arrive at longer (laser) wavelengths.

As can be derived from the below, the term “laser light source” may also refer to a plurality of (different or identical) laser light sources. In specific embodiments, the term “laser light source” may refer to a plurality N of (identical) laser light sources. In embodiments, N=2, or more. In specific embodiments, N may be at least 5, such as especially at least 8. In this way, a higher brightness may be obtained. In embodiments, laser light sources may be arranged in a laser bank (see also above). The laser bank may in embodiments comprise heat sinking and/or optics e.g. a lens to collimate the laser light. Hence, in embodiments lasers in a laser bank (or “laser array bank”) may share the same optics.

The laser light source is configured to generate laser light source light (or “laser light”). The light source light may essentially consist of the laser light source light. The light source light may also comprise laser light source light of two or more (different or identical) laser light sources. For instance, the laser light source light of two or more (different or identical) laser light sources may be coupled into a light guide, to provide a single beam of light comprising the laser light source light of the two or more (different or identical) laser light sources. In specific embodiments, the light source light is thus especially collimated light source light. In yet further embodiments, the light source light is especially (collimated) laser light source light.

The laser light source light may in embodiments comprise one or more bands, having band widths as known for lasers. In specific embodiments, the band(s) may be relatively sharp line(s), such as having full width half maximum (FWHM) in the range of less than 20 nm at RT, such as equal to or less than 10 nm. Hence, the light source light has a spectral power distribution (intensity on an energy scale as function of the wavelength) which may comprise one or more (narrow) bands.

The beams (of light source light) may be focused or collimated beams of (laser) light source light. The term “focused” may especially refer to converging to a small spot. This small spot may be at the discrete converter region, or (slightly) upstream thereof or (slightly) downstream thereof. Especially, focusing and/or collimation may be such that the cross-sectional shape (perpendicular to the optical axis) of the beam at the discrete converter region (at the side face) is essentially not larger than the cross-section shape (perpendicular to the optical axis) of the discrete converter region (where the light source light irradiates the discrete converter region). Focusing may be executed with one or more optics, like (focusing) lenses. Especially, two lenses may be applied to focus the laser light source light. Collimation may be executed with one or more (other) optics, like collimation elements, such as lenses and/or parabolic mirrors. In embodiments, the beam of (laser) light source light may be relatively highly collimated, such as in embodiments ≤2° (FWHM), more especially ≤1° (FWHM), most especially ≤0.5° (FWHM). Hence, ≤2° (FWHM) may be considered (highly) collimated light source light. Optics may be used to provide (high) collimation (see also above).

The term “solid state material laser”, and similar terms, may refer to a solid state laser like based on a crystalline or glass body dopes with ions, like transition metal ions and/or lanthanide ions, to a fiber laser, to a photonic crystal laser, to a semiconductor laser, such as e.g. a vertical cavity surface-emitting laser (VCSEL), etc.

The term “solid state light source”, and similar terms, may especially refer to semiconductor light sources, such as a light emitting diode (LED), a diode laser, or a superluminescent diode. Instead of the term “solid state light source” also the term “semiconductor-based light source” may be applied. Hence, the term “semiconductor-based light source” may e.g. refer to one or more of a light emitting diode (LED), a diode laser, and a superluminescent diode.

Hence, the light generating device may comprise one or more of a light emitting diode (LED), a diode laser, and a superluminescent diode.

Superluminescent diodes are known in the art. A superluminescent diode may be indicated as a semiconductor device which may be able to emit low-coherence light of a broad spectrum like an LED, while having a brightness in the order of a laser diode.

US2020192017 indicates for instance that “With current technology, a single SLED is capable of emitting over a bandwidth of, for example, at most 50-70 nm in the 800-900 nm wavelength range with sufficient spectral flatness and sufficient output power. In the visible range used for display applications, i.e. in the 450-650 nm wavelength range, a single SLED is capable of emitting over bandwidth of at most 10-30 nm with current technology. Those emission bandwidths are too small for a display or projector application which requires red (640 nm), green (520 nm) and blue (450 nm), i.e. RGB, emission”. Further, superluminescent diodes are amongst others described, in “Edge Emitting Laser Diodes and Superluminescent Diodes”, Szymon Stanczyk, Anna Kafar, Dario Schiavon, Stephen Najda, Thomas Slight, Piotr Perlin, Book Editor(s): Fabrizio Roccaforte, Mike Leszczynski, First published: 3 Aug. 2020 https://doi.org/10.1002/9783527825264.ch9 in chapter 9.3 superluminescent diodes. This book, and especially chapter 9.3, are herein incorporated by reference. Amongst others, it is indicated therein that the superluminescent diode (SLD) is an emitter, which combines the features of laser diodes and light-emitting diodes. SLD emitters utilize the stimulated emission, which means that these devices operate at current densities similar to those of laser diodes. The main difference between LDs and SLDs is that in the latter case, the device waveguide may be designed in a special way preventing the formation of a standing wave and lasing. Still, the presence of the waveguide ensures the emission of a high-quality light beam with high spatial coherence of the light, but the light is characterized by low time coherence at the same time” and “Currently, the most successful designs of nitride SLD are bent, curved, or tilted waveguide geometries as well as tilted facet geometries, whereas in all cases, the front end of the waveguide meets the device facet in an inclined way, as shown in FIG. 9.10. The inclined waveguide suppresses the reflection of light from the facet to the waveguide by directing it outside to the lossy unpumped area of the device chip”. Hence, an SLD may especially be a semiconductor light source, where the spontaneous emission light is amplified by stimulated emission in the active region of the device. Such emission is called “super luminescence”. Superluminescent diodes combine the high power and brightness of laser diodes with the low coherence of conventional light-emitting diodes. The low (temporal) coherence of the source has advantages that the speckle is significantly reduced or not visible, and the spectral distribution of emission is much broader compared to laser diodes, which can be better suited for lighting applications. Especially, with varying electrical current, the spectral power distribution of the superluminescent diode may vary. In this way the spectral power distribution can be controlled, see e.g. also Abdullah A. Alatawi, et al., Optics Express Vol. 26, Issue 20, pp. 26355-26364, https://doi.org/10.1364/OE.26.026355.

As indicated above, the first light generating device may especially be configured to generate first device light. Further, the first light generating device may comprise one or more of a superluminescent diode and a solid state laser. Therefore, the first light generating device may comprise a light source selected from one or more of a superluminescent diode and a solid state laser. The solid state laser may especially comprise a diode laser. In embodiments, the first device light may have one or more wavelengths in the visible (i.e. spectral power at one or more wavelengths in the visible wavelength range). Especially, the first device light may comprise blue light. More especially, the first device light may be blue light. However, other options are herein not excluded.

The terms “visible”, “visible light” or “visible emission” and similar terms refer to light having one or more wavelengths in the range of about 380-780 nm. Herein, UV may especially refer to a wavelength selected from the range of 190-380 nm, such as 200-380 nm. The terms “light” and “radiation” are herein interchangeably used, unless clear from the context that the term “light” only refers to visible light. The terms “light” and “radiation” may thus refer to UV radiation, visible light, and IR radiation. In specific embodiments, especially for lighting applications, the terms “light” and “radiation” refer to (at least) visible light.

The terms “violet light” or “violet emission”, and similar terms, may especially relate to light having a wavelength in the range of about 380-440 nm. In specific embodiments, the violet light may have a centroid wavelength in the 380-440 nm range. The terms “blue light” or “blue emission”, and similar terms, may especially relate to light having a wavelength in the range of about 440-490 nm (including some violet and cyan hues). In specific embodiments, the blue light may have a centroid wavelength in the 440-490 nm range. The terms “green light” or “green emission”, and similar terms, may especially relate to light having a wavelength in the range of about 490-560 nm. In specific embodiments, the green light may have a centroid wavelength in the 490-560 nm range. The terms “yellow light” or “yellow emission”, and similar terms, may especially relate to light having a wavelength in the range of about 560-590 nm. In specific embodiments, the yellow light may have a centroid wavelength in the 560-590 nm range. The terms “orange light” or “orange emission”, and similar terms, may especially relate to light having a wavelength in the range of about 590-620 nm. In specific embodiments, the orange light may have a centroid wavelength in the 590-620 nm range. The terms “red light” or “red emission”, and similar terms, may especially relate to light having a wavelength in the range of about 620-750 nm. In specific embodiments, the red light may have a centroid wavelength in the 620-750 nm range. The terms “cyan light” or “cyan emission”, and similar terms, especially relate to light having a wavelength in the range of about 490-520 nm. In specific embodiments, the cyan light may have a centroid wavelength in the 490-520 nm range. The terms “amber light” or “amber emission”, and similar terms, may especially relate to light having a wavelength in the range of about 585-605 nm, such as about 590-600 nm. In specific embodiments, the amber light may have a centroid wavelength in the 585-605 nm range. The phrase “light having one or more wavelengths in a wavelength range” and similar phrases may especially indicate that the indicated light (or radiation) has a spectral power distribution with at least intensity or intensities at these one or more wavelengths in the indicate wavelength range. For instance, a blue emitting solid state light source will have a spectral power distribution with intensities at one or more wavelengths in the 440-495 nm wavelength range.

Further, the system may comprise a luminescent body. Here below, first some general aspects in relation to the luminescent body are described.

Especially, the luminescent material is comprised by a luminescent body. The luminescent body may be a layer, like a self-supporting layer. The luminescent body may also comprise a luminescent coating on a support (especially a light transmissive support in the transmissive mode, or a reflective support in the reflective mode). Especially, the luminescent body may essentially be self-supporting. In embodiments, the luminescent material may be provided as luminescent body, such as a luminescent single crystal, a luminescent glass, or a luminescent ceramic body. Such body may be indicated as “converter body” or “luminescent body”. In embodiments, the luminescent body may be a luminescent single crystal or a luminescent ceramic body. For instance, in embodiments a cerium comprising garnet luminescent material may be provided as a luminescent single crystal or as a luminescent ceramic body. In other embodiments, the luminescent body may comprise a light transmissive body, wherein the luminescent material is embedded. For instance, the luminescent body may comprise a glass body, with luminescent material embedded therein. Or, the glass as such may be luminescent. In other embodiments, the luminescent body may comprise a polymeric body, with luminescent material embedded therein.

The luminescent body may have any shape. In general, however, the luminescent body may comprise two essentially parallel faces, defining a height (of the luminescent body). Further, the luminescent body may comprise an edge face, bridging the two essentially parallel faces. The edge face may be curved in one or two dimensions. The edge face may be planar. The luminescent body may have a rectangular or circular cross-section, though other cross-sections may also be possible, like e.g. hexagonal, octagonal, etc. Hence, the luminescent body may have a circular cross-section, an oval cross-section, square, or non-square rectangular. In embodiments, the luminescent body may have an n-gonal cross-section, wherein n is at least 3, like 4 (square or rectangular cross-section), 5 (pentagonal cross-section), 6 (hexagonal cross-section), 8 (octagonal cross-section) or higher. The two essentially parallel faces may also be indicated as “main faces”, as they may especially provide the largest external area of the luminescent body. Perpendicular to the afore-mentioned cross-section, may be another cross-section, which may in embodiments be rectangular. Hence, the luminescent body may e.g. have a cubic shape, a (non-cubic) cuboid shape, an n-gonal prism shape with n being at least 5 (such as pentagonal prism, hexagonal prism), and a cylindrical shape. Other shapes, however, may also be possible. Especially, the luminescent body may have a cuboid shape, a cylindrical shape, or an n-gonal prism shape wherein n is 6 or 8.

In embodiments, the luminescent body (or “body”) has lateral dimensions width or length (W1 or L1) or diameter (D) and a thickness or height (H1). In embodiments, (i) D≥H1 or (ii) and W1≥H1 and/or L1≥H1. The luminescent body may be transparent or light scattering. In embodiments, the luminescent body may comprise a ceramic luminescent material. In specific embodiments, L1≤10 mm, such as especially L1≤5 mm, more especially L1≤3 mm, most especially L1≤2 mm. In specific embodiments, W1≤10 mm, such as especially W1≤5 mm, more especially W1≤3 mm, most especially W1≤2 mm. In specific embodiments. H1≤10 mm, such as especially H1≤5 mm, more especially H1≤3 mm, most especially H1≤2 mm. In specific embodiments, D≤10 mm, such as especially D≤5 mm, more especially D≤3 mm, most especially D≤2 mm. In specific embodiments, the body may have in embodiments a thickness in the range 50 μm-1 mm. Further, the body may have lateral dimensions (width/diameter) in the range 100 μm-10 mm. In yet further specific embodiments, (i) D>H1 or (ii) W1>H1 and L1>H1. Especially, the lateral dimensions like length, width, and diameter are at least 2 times, like at least 5 times, larger than the height. In specific embodiments, the luminescent body has a first length L1, a first height H1, and a first width W1, wherein H1≤0.5*L1 and H1≤0.5*W1. In embodiments, the luminescent body may be a (small) tile.

In embodiments, the luminescent body may comprises a first face, a second face, and a side face bridging the first face and the second face. The first face and the second face may also be indicated as main faces. In the case of a cylindrical shape, the side face may be a single side face. In the case of a cuboid, the side face may comprise four facets. In the case of a hexagonal prism the side face may comprise six facets.

In specific embodiments, the luminescent body has an annular shape. Hence, in embodiments the luminescent body may have a hollow cylindrical shape. The hollow cylinder may have a height, which may be substantially constant over the ring, and which may substantially be independent of the radius. The annular shaped luminescent body may have an inner radius and an outer radius, defining a width of the hollow cylindrical body. The width may be essentially the same over the entire luminescent body. The inner radius and the outer radius may be larger than the height, such as each at least twice as large as the height.

Further, the luminescent body may especially comprises a luminescent material. Here below, some embodiments of luminescent materials are described.

The term “luminescent material” especially refers to a material that can convert first radiation, especially one or more of UV radiation and blue radiation, into second radiation. In general, the first radiation and second radiation have different spectral power distributions. Hence, instead of the term “luminescent material”, also the terms “luminescent converter” or “converter” may be applied. In general, the second radiation has a spectral power distribution at larger wavelengths than the first radiation, which is the case in the so-called down-conversion. In specific embodiments, however the second radiation has a spectral power distribution with intensity at smaller wavelengths than the first radiation, which is the case in the so-called up-conversion.

In embodiments, the “luminescent material” may especially refer to a material that can convert radiation into e.g. visible and/or infrared light. For instance, in embodiments the luminescent material may be able to convert one or more of UV radiation and blue radiation, into visible light. The luminescent material may in specific embodiments also convert radiation into infrared radiation (IR). Hence, upon excitation with radiation, the luminescent material emits radiation. In general, the luminescent material will be a down converter, i.e. radiation of a smaller wavelength is converted into radiation with a larger wavelength (λₑₓ<λₑₘ), though in specific embodiments the luminescent material may comprise up-converter luminescent material, i.e. radiation of a larger wavelength is converted into radiation with a smaller wavelength (λₑₓ>λₑₘ).

In embodiments, the term “luminescence” may refer to phosphorescence. In embodiments, the term “luminescence” may also refer to fluorescence. Instead of the term “luminescence”, also the term “emission” may be applied. Hence, the terms “first radiation” and “second radiation” may refer to excitation radiation and emission (radiation), respectively. Likewise, the term “luminescent material” may in embodiments refer to phosphorescence and/or fluorescence.

The term “luminescent material” may also refer to a plurality of different luminescent materials. Examples of possible luminescent materials are indicated below. Hence, the term “luminescent material” may in specific embodiments also refer to a luminescent material composition. Instead of the term “luminescent material” also the term “phosphor” may be applied. These terms are known to the person skilled in the art.

In embodiments, luminescent materials are selected from garnets and nitrides, especially doped with trivalent cerium or divalent europium, respectively. The term “nitride” may also refer to oxynitride or nitridosilicate, etc. Alternatively or additionally, the luminescent material(s) may be selected from silicates, especially doped with divalent europium.

In specific embodiments the luminescent material comprises a luminescent material of the type A₃B₅O₁₂:Ce, wherein A in embodiments comprises one or more of Y, La, Gd, Tb and Lu, especially (at least) one or more of Y, Gd, Tb and Lu, and wherein B in embodiments comprises one or more of Al, Ga, In and Sc. Especially, A may comprise one or more of Y, Gd and Lu, such as especially one or more of Y and Lu. Especially, B may comprise one or more of Al and Ga, more especially at least Al, such as essentially entirely Al. Hence, especially suitable luminescent materials are cerium comprising garnet materials. Embodiments of garnets especially include A₃B₅O₁₂ garnets, wherein A comprises at least yttrium or lutetium and wherein B comprises at least aluminum. Such garnets may be doped with cerium (Ce), with praseodymium (Pr) or a combination of cerium and praseodymium; especially however with Ce. Especially, B may comprise aluminum (Al); however, in addition to aluminum, B may also partly comprise gallium (Ga) and/or scandium (Sc) and/or indium (In), especially up to about 20% of B, more especially up to about 10% of B (i.e. the B ions essentially consist of 90 or more mole % of Al and 10 or less mole % of one or more of Ga, Sc and In); B may especially comprise up to about 10% gallium. In another variant, B and O may at least partly be replaced by Si and N. The element A may especially be selected from the group consisting of yttrium (Y), gadolinium (Gd), terbium (Tb) and lutetium (Lu). Further, Gd and/or Tb are especially only present up to an amount of about 20% of A. In a specific embodiment, the garnet luminescent material comprises (Y₁₋ₓLuₓ)₃B₅O₁₂:Ce, wherein x is equal to or larger than 0 and equal to or smaller than 1. The term “:Ce”, indicates that part of the metal ions (i.e. in the garnets: part of the “A” ions) in the luminescent material is replaced by Ce. For instance, in the case of (Y₁₋ₓLuₓ)₃Al₅O₁₂:Ce, part of Y and/or Lu is replaced by Ce. This is known to the person skilled in the art. Ce will replace A in general for not more than 10%; in general, the Ce concentration will be in the range of 0.1 to 4%, especially 0.1 to 2% (relative to A). Assuming 1% Ce and 10% Y, the full correct formula could be (Y_{0.1}Lu_{0.89}Ce_{0.01})₃Al₅O₁₂. Ce in garnets is substantially or only in the trivalent state, as is known to the person skilled in the art.

In embodiments, the luminescent material (thus) comprises A₃B₅O₁₂ wherein in specific embodiments at maximum 10% of B—O may be replaced by Si—N.

In specific embodiments the luminescent material comprises (Yₓ₁₋ₓ₂₋ₓ₃A′ₓ₂Ceₓ₃)₃(Al_{y1-y2}B′_{y2})₅O₁₂, wherein x1+x2+x3=1, wherein x3>0, wherein 0<x2+x3≤0.2, wherein y1+y2=1, wherein 0≤y2≤0.2, wherein A′ comprises one or more elements selected from the group consisting of lanthanides, and wherein B′ comprises one or more elements selected from the group consisting of Ga, In and Sc. In embodiments, x3 is selected from the range of 0.001-0.1. In the present invention, especially x1>0, such as >0.2, like at least 0.8. Garnets with Y may provide suitable spectral power distributions.

In specific embodiments at maximum 10% of B—O may be replaced by Si—N. Here, B in B—O refers to one or more of Al, Ga, In and Sc (and O refers to oxygen); in specific embodiments B—O may refer to Al—O. As indicated above, in specific embodiments ×3 may be selected from the range of 0.001-0.04. Especially, such luminescent materials may have a suitable spectral distribution (see however below), have a relatively high efficiency, have a relatively high thermal stability, and allow a high CRI (optionally in combination with (the) light of other sources of light as described herein). Hence, in specific embodiments A may be selected from the group consisting of Lu and Gd. Alternatively or additionally, B may comprise Ga. Hence, in embodiments the luminescent material comprises (Yₓ₁₋ₓ₂₋ₓ₃(Lu,Gd)ₓ₂Ceₓ₃)₃(Al_{y1-y2}Ga_{y2})₅O₁₂, wherein Lu and/or Gd may be available. Even more especially, x3 is selected from the range of 0.001-0.1, wherein 0<x2+x3≤0.1, and wherein 0≤y2≤0.1. Further, in specific embodiments, at maximum 1% of B—O may be replaced by Si—N. Here, the percentage refers to moles (as known in the art); see e.g. also EP3149108. In yet further specific embodiments, the luminescent material comprises (Yₓ₁₋ₓ₃Ceₓ₃)₃Al₅O₁₂, wherein x1+x3=1, and wherein 0<x3≤0.2, such as 0.001-0.1.

In specific embodiments, the light generating device may only include luminescent materials selected from the type of cerium comprising garnets. In even further specific embodiments, the light generating device includes a single type of luminescent materials, such as (Yₓ₁₋ₓ₂₋ₓ₃A′ₓ₂Ceₓ₃)₃(Al_{y1-y2}B′_{y2})₅O₁₂. Hence, in specific embodiments the light generating device comprises luminescent material, wherein at least 85 weight %, even more especially at least about 90 wt. %, such as yet even more especially at least about 95 weight % of the luminescent material comprises (Yₓ₁₋ₓ₂₋ₓ₃A′ₓ₂Ceₓ₃)₃(Al_{y1-y2}B′_{y2})₅O₁₂. Here, wherein A′ comprises one or more elements selected from the group consisting of lanthanides, and wherein B′ comprises one or more elements selected from the group consisting of Ga, In and Sc, wherein x1+x2+x3=1, wherein x3>0, wherein 0<x2+x3≤0.2, wherein y1+y2=1, wherein 0≤y2<0.2. Especially, x3 is selected from the range of 0.001-0.1. Note that in embodiments x2=0. Alternatively or additionally, in embodiments y2=0.

In specific embodiments, A may especially comprise at least Y, and B may especially comprise at least Al.

Alternatively or additionally, wherein the luminescent material may comprises a luminescent material of the type A₃Si₆N₁₁:Ce³⁺, wherein A comprises one or more of Y, La, Gd, Tb and Lu, such as in embodiments one or more of La and Y.

In embodiments, the luminescent material may alternatively or additionally comprise one or more of MS:Eu²⁺ and/or M₂Si₅N₈:Eu²⁺ and/or MAlSiN₃:Eu²⁺ and/or Ca₂AlSi₃O₂N₅:Eu²⁺, etc., wherein M comprises one or more of Ba, Sr and Ca, especially in embodiments at least Sr. Hence, in embodiments, the luminescent may comprise one or more materials selected from the group consisting of (Ba,Sr,Ca)S:Eu, (Ba,Sr,Ca)AlSiN₃:Eu and (Ba,Sr,Ca)₂Si₅N₈:Eu. In these compounds, europium (Eu) is substantially or only divalent, and replaces one or more of the indicated divalent cations. In general, Eu will not be present in amounts larger than 10% of the cation; its presence will especially be in the range of about 0.5 to 10%, more especially in the range of about 0.5 to 5% relative to the cation(s) it replaces. The term “:Eu”, indicates that part of the metal ions is replaced by Eu (in these examples by Eu²⁺). For instance, assuming 2% Eu in CaAlSiN₃:Eu, the correct formula could be (Ca_{0.98}Eu_{0.02})AlSiN₃. Divalent europium will in general replace divalent cations, such as the above divalent alkaline earth cations, especially Ca, Sr or Ba. The material (Ba,Sr,Ca)S:Eu can also be indicated as MS:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca). Further, the material (Ba,Sr,Ca)₂Si₅N₈:Eu can also be indicated as M₂Si₅N₈:Eu, wherein M is one or more elements selected from the group consisting of barium (Ba), strontium (Sr) and calcium (Ca); especially, M comprises in this compound Sr and/or Ba. In a further specific embodiment, M consists of Sr and/or Ba (not taking into account the presence of Eu), especially 50 to 100%, more especially 50 to 90% Ba and 50 to 0%, especially 50 to 10% Sr, such as Ba_{1.5}Sr_{0.5}Si₅N₈:Eu (i.e. 75% Ba; 25% Sr). Here, Eu is introduced and replaces at least part of M, i.e. one or more of Ba, Sr, and Ca). Likewise, the material (Ba,Sr,Ca)AlSiN₃:Eu can also be indicated as MAlSiN₃:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca). Eu in the above indicated luminescent materials is substantially or only in the divalent state, as is known to the person skilled in the art.

In embodiments, a red luminescent material may comprise one or more materials selected from the group consisting of (Ba,Sr,Ca)S:Eu, (Ba,Sr,Ca)AlSiN₃:Eu and (Ba,Sr,Ca)₂Si₅N₈:Eu. In these compounds, europium (Eu) is substantially or only divalent, and replaces one or more of the indicated divalent cations. In general, Eu will not be present in amounts larger than 10% of the cation; its presence will especially be in the range of about 0.5 to 10%, more especially in the range of about 0.5 to 5% relative to the cation(s) it replaces. The term “:Eu”, indicates that part of the metal ions is replaced by Eu (in these examples by Eu²⁺). For instance, assuming 2% Eu in CaAlSiN₃:Eu, the correct formula could be (Ca_{0.98}Eu_{0.02})AlSiN₃. Divalent europium will in general replace divalent cations, such as the above divalent alkaline earth cations, especially Ca, Sr or Ba.

The material (Ba,Sr,Ca)S:Eu can also be indicated as MS:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca).

Further, the material (Ba,Sr,Ca)₂Si₅N₈:Eu can also be indicated as M₂Si₅N₈:Eu, wherein M is one or more elements selected from the group consisting of barium (Ba), strontium (Sr) and calcium (Ca); especially, M comprises in this compound Sr and/or Ba. In a further specific embodiment, M consists of Sr and/or Ba (not taking into account the presence of Eu), especially 50 to 100%, more especially 50 to 90% Ba and 50 to 0%, especially 50 to 10% Sr, such as Ba_{1.5}Sr_{0.5}Si₅N₈:Eu (i.e. 75% Ba; 25% Sr). Here, Eu is introduced and replaces at least part of M, i.e. one or more of Ba, Sr, and Ca).

Likewise, the material (Ba,Sr,Ca)AlSiN₃:Eu can also be indicated as MAlSiN₃:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca).

Eu in the above indicated luminescent materials is substantially or only in the divalent state, as is known to the person skilled in the art.

Blue luminescent materials may comprise YSO (Y₂SiO₅:Ce³⁺), or similar compounds, or BAM (BaMgAl₁₀O₁₇:Eu²⁺), or similar compounds.

The term “luminescent material” herein especially relates to inorganic luminescent materials.

Alternatively or additionally, also other luminescent materials may be applied. For instance quantum dots and/or organic dyes may be applied and may optionally be embedded in transmissive matrices like e.g. polymers, like PMMA, or polysiloxanes, etc. etc.

Quantum dots are small crystals of semiconducting material generally having a width or diameter of only a few nanometers. When excited by incident light, a quantum dot emits light of a color determined by the size and material of the crystal. Light of a particular color can therefore be produced by adapting the size of the dots. Most known quantum dots with emission in the visible range are based on cadmium selenide (CdSe) with a shell such as cadmium sulfide (CdS) and zinc sulfide (ZnS). Cadmium free quantum dots such as indium phosphide (InP), and copper indium sulfide (CuInS₂) and/or silver indium sulfide (AgInS₂) can also be used. Quantum dots show very narrow emission band and thus they show saturated colors. Furthermore the emission color can easily be tuned by adapting the size of the quantum dots. Any type of quantum dot known in the art may be used in the present invention. However, it may be preferred for reasons of environmental safety and concern to use cadmium-free quantum dots or at least quantum dots having a very low cadmium content.

Instead of quantum dots or in addition to quantum dots, also other quantum confinement structures may be used. The term “quantum confinement structures” should, in the context of the present application, be understood as e.g. quantum wells, quantum dots, quantum rods, tripods, tetrapods, or nano-wires, etcetera.

Organic phosphors can be used as well. Examples of suitable organic phosphor materials are organic luminescent materials based on perylene derivatives, for example compounds sold under the name Lumogen® by BASF. Examples of suitable compounds include, but are not limited to, Lumogen® Red F305, Lumogen® Orange F240, Lumogen® Yellow F083, and Lumogen® F170.

Different luminescent materials may have different spectral power distributions of the respective luminescent material light. Alternatively or additionally, such different luminescent materials may especially have different color points (or dominant wavelengths).

As indicated above, other luminescent materials may also be possible. Hence, in specific embodiments the luminescent material is selected from the group of divalent europium containing nitrides, divalent europium containing oxynitrides, divalent europium containing silicates, cerium comprising garnets, and quantum structures. Quantum structures may e.g. comprise quantum dots or quantum rods (or other quantum type particles) (see above). Quantum structures may also comprise quantum wells. Quantum structures may also comprise photonic crystals.

The luminescent material may especially be configured to convert at least part of the first device light into luminescent material light. The luminescent material light may have one or more wavelengths in the visible (i.e. spectral power at one or more wavelengths in the visible wavelength range). Especially, the luminescent material light may comprise one or more of green, yellow, orange, and red light.

Hence, especially the luminescent body may be configured in a light receiving relationship with the light generating device. As indicated below, this may especially be a light receiving relationship via the axicon-like optical element.

The terms “light-receiving relationship” or “light receiving relationship”, and similar terms, may indicate that an item may during operation of a source of light (like a light generating device or light generating element or light generating system) may receive light from that source of light. Hence, the item may be configured downstream of that source of light. Between the source of light and the item, optics may be configured. The terms “upstream” and “downstream”, such as in the context of propagation of light, may especially relate to an arrangement of items or features relative to the propagation of the light from a light generating element (here the especially the light generating device), wherein relative to a first position within a beam of light from the light generating element, a second position in the beam of light closer to the light generating element (than the first position) is “upstream”, and a third position within the beam of light further away from the light generating element (than the first position) is “downstream”. Instead of the term “light generating element” also the term “light generating means” may be applied.

Here below, an optical relation between the luminescent body and the axicon-like optical element is further described. However, first some aspects in relation to the thermally conductive element are described.

The thermally conductive element may especially comprise a thermally conductive material. A thermally conductive material may especially have a thermal conductivity of at least about 20 W/(m*K), like at least about 30 W/(m*K), such as at least about 100 W/(m*K), like especially at least about 200 W/(m*K). In yet further specific embodiments, a thermally conductive material may especially have a thermal conductivity of at least about 10 W/(m*K). In embodiments, the thermally conductive material may comprise one or more of copper, aluminum, silver, gold, silicon carbide, aluminum nitride, boron nitride, aluminum silicon carbide, beryllium oxide, a silicon carbide composite, aluminum silicon carbide, a copper tungsten alloy, a copper molybdenum carbide, carbon, diamond, and graphite. Alternatively, or additionally, the thermally conductive material may comprise or consist of aluminum oxide. In embodiments, the thermally conductive element may comprise one or more of a heatsink, a heat spreader, and a two-phase cooling device. In yet other embodiments, the thermally conductive element may be configured in thermal contact with one or more of a heatsink, a heat spreader, and a two-phase cooling device, and may e.g. transfer heat to such heatsink, heat spreader, or two-phase cooling device, via another thermally conductive element.

An element may be considered in “thermal contact” with another element if it can exchange energy through the process of heat. Hence, the elements may be thermally coupled. In embodiments, thermal contact can be achieved by physical contact. In embodiments, thermal contact may be achieved via a thermally conductive material, such as a thermally conductive glue (or thermally conductive adhesive). Thermal contact may also be achieved between two elements when the two elements are arranged relative to each other at a distance of equal to or less than about 10 μm, though larger distances, such as up to 100 μm may be possible. The shorter the distance, the better the thermal contact. Especially, the distance is 10 μm or less, such as 5 μm or less, such as 1 μm or less. The distance may be the distanced between two respective surfaces of the respective elements. The distance may be an average distance. For instance, the two elements may be in physical contact at one or more, such as a plurality of positions, but at one or more, especially a plurality of other positions, the elements are not in physical contact. For instance, this may be the case when one or both elements have a rough surface. Hence, in embodiments in average the distance between the two elements may be 10 μm or less (though larger average distances may be possible, such as up to 100 μm). In embodiments, the two surfaces of the two elements may be kept at a distance with one or more distance holders. When two elements are in thermal contact, they may be in physical contact or may be configured at a short distance of each other, like at maximum 10 μm, such as at maximum 1 mm. When the two elements are configured at a distance from each other, an intermediate material may be configured in between, though in other embodiments, the distance between the two elements may filled with a gas, liquid, or may be vacuum. When an intermediate material is available, the larger the distance, the higher the thermal conductivity may be useful for thermal contact between the two elements. However, the smaller the distance, the lower the thermal conductivity of the intermediate material may be (of course, higher thermal conductive materials may also be used).

As indicated above, the thermally conductive element may be configured in thermal contact with at least part of the luminescent body. For instance, the luminescent body may be configured in physical contact with the thermally conductive element. Further, the thermally conductive element may be is reflective for one or more of the first device light and the luminescent material light, especially for both.

Herein, when an element is indicated to be transmissive this may in embodiments imply that at one or more wavelengths the part of the radiation that is transmitted may be larger than the part of the radiation that is reflected or absorbed. Herein, when an element is indicated to be reflective this may in embodiments imply that at one or more wavelengths the part of the radiation that is reflected may be larger than the part of the radiation that is transmitted or absorbed. The term “transmissive” with regards to the light source light may herein may especially refer to at least 50% of incident light source light passing through the material, such as at least 60%, especially at least 70%, such as at least 80%, especially at least 90%, such as at least 95%, under perpendicular irradiation. Similarly, the term “reflective” with regards to the light source light may herein refer to at least 50% of incident light source light being reflected, such as at least 60%, especially at least 70%, such as at least 80%, especially at least 90%, such as at least 95%, under perpendicular irradiation. Here, the percentages may refer to percentages based on Watts.

As indicated above, the system may further comprise an axicon like optical element. The axicon-like optical element especially comprises a lens. In other words, the system may comprise a lens which has an axicon-like shape. Axicons lenses are known in the art, and can e.g. be defined as a specialized type of lens which has a conical surface. An axicon lens may especially be able to transforms a laser beam into a ring shaped distribution (see e.g. http://wp.optics.arizona.edu/wp-content/uploads/2016/03/axicon_Proteep.pdf). Herein, the term axicon-like is used to indicate that there may also be small deviations from the axicon shape. For instance, the conical shape may in embodiments be a bit rounded, instead of an essentially pure cone (however, a radius of curvature of the surface of such rounded conical shape should not be too small, for instance it should be larger than the (smallest) diameter of the axicon. Further, herein the axicon-like optical element may have a relatively large cylindrical part. The cylindrical part may be non-tapered or tapered (see also below).

In embodiment, the axicon-like optical element comprises a first part and a second part. Especially, the axicon-like optical element may have an optical axis. The first part may be configured rotationally symmetric relative to the optical axis and the second part may be configured rotationally symmetric relative to the optical axis. Especially, the axicon-like optical element is a monolithic body of a light transparent material, such as quartz, glass, sapphire, polymeric material etc., essentially consisting of the first part and the second part. Further, the axicon-like optical element may have an optical element length (L). Especially, this optical element length may be defined parallel to the optical axis. Should the axicon-like optical element comprise a polymeric light transparent material, such material may e.g. comprise one or more of polycarbonate (PC), silicone (polysiloxane), polystyrene, polymethylmethacrylate, etc. Especially, the axicon-like optical element may be a solid body essentially consisting of the light transparent material.

Especially, the first part may have a conical shape. The conical shape may especially be a cone. Further, the conical shape may have a cone angle selected from the range of about 90-178°, more especially at least about 100°, such as selected from the range of about 105-125°. Further, the first part may have a first length (L1). Especially, this first length may be defined parallel to the optical axis. Yet further, the first part may comprises a first end window. The term “end window” may refer to the fact that this end window may be configured at one end of the axicon-like optical element. Further, this term may indicate that light may especially enter and/or escape via that part of the axicon-like optical element. Especially, the entire surface of the cone may be the end window. Nevertheless, this does not necessarily mean that the entire end window is used as such.

Further, the second part may have a cylindrical shape. Yet further, this part may have a second length (L2). Especially, this second length may be defined parallel to the optical axis. Further, this first part may comprise a second end window. Especially, this second part may essentially be planar. The term “second end window” may refer to the fact that this end window may be configured at another end of the axicon-like optical element. Further, this term may indicate that light may especially enter and/or escape via that part of the axicon-like optical element. Especially, the entire end surface of the cylinder may be the (second) end window. Nevertheless, this does not necessarily mean that the entire end window is used as such.

In relation to the axicon-like optical element the first part and the second part, i.e., essentially the entire axicon-like optical element, may especially have a circular cross-section (perpendicular to the optical axis). However, other cross-sectional shapes are herein not excluded, such as hexagonal, octagonal, decagonal, and higher gonal shapes.

The optical element length (L) may be defined by the first end window, especially the apex, and the second end window. Hence, especially L=L1+L2. Further, herein especially L1<L2. More especially, in embodiments 0.6≤L2/L<1, more especially 0.7≤L2/L<1, such as 0.75≤L2/L<0.95. Further, in specific embodiments 0.8≤L2/L<1, more especially 0.8≤L2/L≤0.95. In embodiments, L2/L may be selected from the range of 0.8-0.02. Especially, these shapes and dimensions may allow a dual function of the axicon-like optical element, as further also described below.

Especially, the axicon-like optical element may be used to project light of the light generating device on the luminescent body, but may also be used to collect luminescent material light from the luminescent body and provide a beam of luminescent material light. Hence, in embodiments first device light enters via the first end window of the axicon-like optical element but luminescent material light may also escape from the axicon-like optical element via this window. Similarly, first device light may, after propagation through the axicon-like optical element, optionally including (total) internal reflection, via the second end window of the axicon-like optical element, but luminescent material light may also enter the axicon-like optical element via this second end window (and propagate through the axicon-like optical element, optionally including (total) internal reflection, to the first end window of the axicon-like optical element, and escape via this first end window).

Therefore, in specific embodiments the axicon-like optical element may be configured to: (a) receive at least part of the first device light via the first part (especially the first end window) and provide an annular beam of first device light via the second part (especially the second end window) to the luminescent body, and (b) collect at least part of the luminescent material light via the second part (especially the second end window) and provide a beam of luminescent material light via the first part (especially the first end window).

As indicated above, this may provide a relatively compact high intensity source of light. Especially, in this way an annular shaped phosphor in combination with axicon lens for producing laser pumped high intensity light source may be provided. As further elucidated below, in specific embodiments this may be a laser pumped high intensity white light source.

Further embodiments will be described below.

As indicated above, the cylindrical part may be non-tapered (“untapered”) or tapered (see also below). Especially, in embodiments the second part may taper over at least part of the second length (L2) in a direction from the first part to the second end window. This tapering may especially be useful in view of the collimation of the luminescent material light. Hence, the smallest diameter of axicon-like optical element may in embodiments be defined by the second end window. The smallest diameter of the second part may thus in embodiments be the same as the largest diameter of the first part (non-tapered), or may be smaller (tapered). Especially, the ratio of the diameters is not smaller than 0.5. Therefore, in embodiments the first end window may have a largest first window radius (Rw1), the second end window may have a second window radius (R_{W2}), wherein in embodiments RW2/RW1=1 (non-tapered), or wherein in other embodiments RW2/RW1<1. In specific embodiments, 0.5≤RW2/RW1≤0.98. Especially, these shapes and dimensions may allow a dual function of the axicon-like optical element, as further also described below.

A non-tapered cylinder may have a constant diameter and a tapered cylinder or cone may have a constantly decreasing or increasing diameter (increasing or decreasing over its height).

The light generating system may especially be configured system light. The system light may comprise at least part of the luminescent material light that has escaped from the first end window. Such luminescent material light may especially be non-white light (see also above). For white light applications, it may be desirable to provide one or more sources of light which may contribute to the system light, and provide white light, with a desirable correlated color temperature (CCT) and/or a desirable color rendering index (CRI). Several solutions appear possible, such as using a source of light bypassing the optical element and the luminescent material. However, the pump light as such may also be used as component of the system light. This may especially be the case when part of the first device light is reflected at the luminescent body and enters the axicon-like optical element again via the second end window. Then, the light emanating from the first end window may comprise both the luminescent material light and part of the first device light. By choosing the axicon-like optical element and the radii of the luminescent body, the part of the first device light that may only be reflected at the thermally conductive element and may have essentially no interaction with the luminescent body may be controlled.

In embodiments, in the range of 0-35%, such as in the range of about 2-35%, like in the range of 2-30% of the spectral power of the first device light escaping from the second end window may be reflected at the thermally conductive body, either without any interaction with the luminescent body or after transmission through the luminescent body (i.e. propagating twice the optical path length), and reach the second end window again as unconverted and reflected first device light. Here, the percentage refer to the percentage relative to the spectral power in watt. In embodiments, up to about 70% thereof (i.e. at maximum 70% of 35% (or of 30%, respectively)), may have had no interaction at all with the luminescent body, such as up to about 50%. Hence, in embodiments, the light generating system may be configured such that part of the first device light escaping from the second end window may be reflected at one or more of the thermally conductive element and the luminescent body, enters after reflection the axicon-like optical element via the second end window, and escapes, together with at least part of the luminescent material light, via the first end window from the axicon-like optical element, to provide a beam of light comprising first device light and the luminescent material light. Hence, the system light may comprise at least part of the first device light and at least part of the luminescent material light. In this way, optionally in combination with one of more further sources of light, white system light may be provided with the light generating system.

Light escaping from the first end window and first device light irradiating the first end window may have essentially parallel optical axis. Hence, an optical element directing the first device light into the first part and/or an optical element directing the luminescent material light away from the optical axis of the axicon-like optical element may in embodiments be relatively small compared to the beam of luminescent material light escaping from the first end window and/or have dichroic functionality. The former solution may be useful when also reflected first device light may have to propagate along the luminescent material light end be part of the system light. The latter solution may be useful when such first device light is not desirable. A combination of solutions may also be applied. Such optics are herein also indicated as “first optics”.

Hence, in embodiments the system may comprise optional first optics. In embodiments, the first optics may be configured in the optical pathway of the first device light between the first light generating device and the first end window. The first optics may be configured to reflect the first device light such that after reflection it irradiates the first end window with the first device light. In such embodiments the first optics may comprise a reflector or a dichroic mirror, with the latter being transmissive for the luminescent material light and reflective for the first device light. In alternative embodiments, not further discussed here, the first optics may comprise a dichroic mirror being reflective for the luminescent material light and transmissive for the first device light.

In embodiments the first end window has a largest first window circular cross-section (A_{w1}). Further, as indicated above, the optional first optics may comprise a reflector. In specific embodiments, the reflector may in specific embodiments comprise a dichroic mirror (reflective for the first device light and transmissive for the luminescent material light). The reflector, such as in embodiments the dichroic mirror, may have an optics cross-section (Aₒ₁), defined parallel to the largest first window circular cross-section (A_{w1}). Especially, Aₒ₁/A_{w1}≤0.5. In this way, the beam of luminescent material light may not be too much intercepted by the reflector. In embodiments, 0.01≤Aₒ₁/A_{w1}≤0.5. Other values, however, may also be possible. Even values larger than 0.5 may be possible, when the reflector is at substantial distance from the axicon-like optical element. However, this may be less desirable when smaller sizes of the system are desirable.

It appears useful when the first device light does not fully irradiate the first end window, but only part around the optical axis of the axicon-like optical element. In embodiments, the first light generating device and optional first optics are configured to provide a beam of first device light at the first end window having a pump beam circular cross-section (Aₚ). Further, in embodiments the first end window has a largest first window circular cross-section (A_{w1}). Especially, in embodiments Aₚ/A_{w1}≤0.8.

As indicated above, the luminescent body may be configured in thermal contact with the thermally conductive body (see further also below). In specific embodiments, the luminescent body may also be configured in thermal contact with the axicon-like optical element. Hence, basically, there may be the following options: (a) the luminescent body and the axicon like optical element are not in optical contact and not in thermal contact, (b) they are in thermal contact, but not in optical contact, (c) they are both in thermal contact and optical contact, but not in physical contact, and (d) they are in thermal contact, optical contact, and physical contact.

Thermal contact has been described above. When elements are in optical contact or optically coupled, they may in embodiments be in physical contact with each other or may in other embodiments be separated from each other with e.g. a (thin) layer of optical material, such as an optical glue, or other optically transparent interface material, e.g. having a thickness of less than about 1 mm, preferably less than 100 μm. When no optically transparent interface material is applied, the (average) distance between two elements being in optical contact may especially be about at maximum the wavelength of relevance, such as the wavelength of an emission maximum. For visible wavelengths, this may be less than 1 μm, such as less than 0.7 μm, and for blue even smaller. Hence, when optical coupling is desired, an optically transparent interface material may be applied. In yet other embodiments, when no optically transparent interface material is applied, the average distance between two elements being in optical contact may especially be about at maximum the wavelength of relevance, such as the wavelength of an emission maximum. Hence, when optical contact is desired, there may be physical contact. However, even in such embodiments there may be a non-zero average distance, but then equal to or lower than the wavelength of interest, such as about 700 nm, or even below about 470 nm (for instance in view of the use of the reflected first device light).

In specific embodiments a first distance (d1) between the luminescent body and the second end window may be selected from the range of 0-0.1*L. In yet further specific embodiments, the distance may at least not be larger than 100 μm. In yet further embodiments, the distance may not be larger than about 0.7 μm, such as not larger than about 0.5 μm. Therefore, in specific embodiments the luminescent body and the second end window may be configured in optical contact (and the distance thus may not be larger than about 0.7 μm, such as not larger than about 0.5 μm).

For efficiency reasons, it appears useful when the end window has a (slightly) larger diameter (or radius), than the largest diameter (or radius) of the luminescent body. Especially, the luminescent body has an outer luminescent body radius (rₗₒ) and a luminescent body inner radius (rₗᵢ). Further, especially the second end window has a second window radius (R_{W2}). As will be clear to a person skilled in the art, especially Rₗₒ>rₗᵢ. Further, in embodiments 0.7≤rₗₒ/R_{W2}≤1.05, more especially 0.80≤rₗₒ/R_{W2}≤1. Especially, in embodiments 0.85 rₗₒ/R_{W2}≤1, more especially 0.85≤rₗₒ/R_{W2}≤1, such as 0.85 rₗₒ/R_{W2}≤0.99.

Especially, the luminescent body may have a first face directed to the second end window, side faces, and a bottom face configured farthest away from the second end window. In embodiments, at least the bottom face may at least partly be in thermal contact with the thermally conductive element. More especially, in embodiments the side faces, and the bottom face may be configured in thermal contact with the thermally conductive element. For instance, this may be the case when the thermally conductive element may comprise a slit wherein the luminescent body may at least partly be hosted. Therefore, in embodiments the thermally conductive element may comprise an annular slit hosting at least part of the luminescent body, wherein especially the side faces, and the bottom face may be configured in thermal contact with the thermally conductive element. One or both sides faces may be in thermal contact over part of their height or may be in thermal contact over the entire height.

Hence, in specific embodiments the thermally conductive body may comprise an annular slit have a shape (and dimensions) corresponding to the annular luminescent body.

In specific embodiments, the luminescent material may comprise a luminescent material of the type A₃B₅O₁₂:Ce, wherein A may comprise one or more of Y, La, Gd, Tb and Lu, and wherein B may comprise one or more of Al, Ga, In and Sc (see further also above). Especially such luminescent material may be stable at high pumping powers. As indicated above, the term “luminescent material” may also refer in embodiments to a combination of two or more different luminescent materials.

In specific embodiments, the luminescent body may comprise a ceramic body. ceramic bodies, such as e.g. comprising luminescent material of the type A₃B₅O₁₂:Ce, may especially be useful in terms of thermal management, because of the relatively high thermal conductivity.

In specific embodiments, the first light generating device may comprise a blue (laser) light emitting diode laser.

As indicated above, the system light may comprise the luminescent material light, optionally including first device light. It may be desirable that optical properties of such light in terms of CRI, CCT, and color point, are further adapted and/or are (more) controllable. To that end, one or more further light generating devices (indicated as “second light generating devices”) may be applied.

Such second light generating device may especially neither be configured to provide second light that has to propagate through the axicon-like optical element nor be configured to interact (including being transmitted and/or reflected) by the luminescent body, though neither of these options are herein excluded.

Further, one or more second light generating devices may be configured to provide second device light having essentially the same spectral power distribution as the first device light, but such one or more second light generating devices may be configured to bypass with their second device light the luminescent body. Such one or more second light generating devices may be used to control a ratio of the pump light, especially blue light, and the luminescent material light in the system light. To this end, the system may also comprise a control system (see further also below).

Alternatively or additionally, one or more (other) second light generating devices may be configured to provide second device light having a substantially different spectral power distribution as the first device light. Also such one or more (other) second light generating devices may be configured to bypass with their second device light the luminescent body. Such one or more (other) second light generating devices may be used to control a ratio of the second device light and the luminescent material light in the system light. To this end, the system may also comprise a control system (see further also below).

In both ways, the CCT, CRI, and color point may be further tuned and/or controlled.

Device light from the one or more second light generating devices may be admixed to the luminescent material light (and optional (unconverted) first device light) with a beam combiner. Hence, in specific embodiments the light generating system may further comprise a beam combiner and a second light generating device. The second light generating device may be configured to generate second device light. The beam combiner may be configured to combine the first luminescent material light downstream of the first end window, the optional first device light, and the second device light. In specific embodiments, the second light generating device may be configured to generate second device light having a second spectral power distribution different from a first spectral power distribution of the first device light. Especially, in embodiments the second device light may have intensity in the orange-red wavelength range (i.e. in the wavelength range of 590-750 nm).

In specific embodiments, in an operational mode of the light generating system, the system light may be white light. This white light may be based on the luminescent material light and one or more of the first device light and the second device light.

The term “white light”, and similar terms, herein, is known to the person skilled in the art. It may especially relate to light having a correlated color temperature (CCT) between about 1800 K and 20000 K, such as between 2000 and 20000 K, especially 2700-20000 K, for general lighting especially in the range of about 2000-7000 K, such as in the range of 2700 K and 6500 K. In embodiments, e.g. for backlighting purposes, or for other purposes, the correlated color temperature (CCT) may especially be in the range of about 7000 K and 20000 K. Yet further, in embodiments the correlated color temperature (CCT) is especially within about 15 SDCM (standard deviation of color matching) from the BBL (black body locus), especially within about 10 SDCM from the BBL, even more especially within about 5 SDCM from the BBL.

In specific embodiments, the correlated color temperature (CCT) may be selected from the range of 6000-12000 K, like selected from the range of 7000-12000 K, like at least 8000 K. Yet further, in embodiments the correlated color temperature (CCT) may be selected from the range of 6000-12000 K, like selected from the range of 7000-12000 K, in combination with a CRI of at least 70.

As indicated above, in specific embodiments the system may further comprise a control system (or the system may be functionally coupled to a control system). The control system may be configured to control the system light, more especially its optical properties. The control system may control the system light by controlling the one or more first light generating devices and the one or more second light generating devices.

The term “controlling” and similar terms especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc. Beyond that, the term “controlling” and similar terms may additionally include monitoring. Hence, the term “controlling” and similar terms may include imposing behavior on an element and also imposing behavior on an element and monitoring the element. The controlling of the element can be done with a control system, which may also be indicated as “controller”. The control system and the element may thus at least temporarily, or permanently, functionally be coupled. The element may comprise the control system. In embodiments, the control system and element may not be physically coupled. Control can be done via wired and/or wireless control. The term “control system” may also refer to a plurality of different control systems, which especially are functionally coupled, and of which e.g. one control system may be a master control system and one or more others may be slave control systems. A control system may comprise or may be functionally coupled to a user interface.

The control system may also be configured to receive and execute instructions from a remote control. In embodiments, the control system may be controlled via an App on a device, such as a portable device, like a Smartphone or I-phone, a tablet, etc. The device is thus not necessarily coupled to the lighting system, but may be (temporarily) functionally coupled to the lighting system.

Hence, in embodiments the control system may (also) be configured to be controlled by an App on a remote device. In such embodiments the control system of the lighting system may be a slave control system or control in a slave mode. For instance, the lighting system may be identifiable with a code, especially a unique code for the respective lighting system. The control system of the lighting system may be configured to be controlled by an external control system which has access to the lighting system on the basis of knowledge (input by a user interface of with an optical sensor (e.g. QR code reader) of the (unique) code. The lighting system may also comprise means for communicating with other systems or devices, such as on the basis of Bluetooth, Thread, WIFI, LiFi, ZigBee, BLE or WiMAX, or another wireless technology.

The system, or apparatus, or device may execute an action in a “mode” or “operation mode” or “mode of operation” or “operational mode”. The term “operational mode may also be indicated as “controlling mode”. Likewise, in a method an action or stage, or step may be executed in a “mode” or “operation mode” or “mode of operation” or “operational mode”. This does not exclude that the system, or apparatus, or device may also be adapted for providing another controlling mode, or a plurality of other controlling modes. Likewise, this may not exclude that before executing the mode and/or after executing the mode one or more other modes may be executed.

However, in embodiments a control system may be available, that is adapted to provide at least the controlling mode. Would other modes be available, the choice of such modes may especially be executed via a user interface, though other options, like executing a mode in dependence of a sensor signal or a (time) scheme, may also be possible. The operation mode may in embodiments also refer to a system, or apparatus, or device, which can only operate in a single operation mode (i.e. “on”, without further tunability).

Hence, in embodiments, the control system may control in dependence of one or more of an input signal of a user interface, a sensor signal (of a sensor), and a timer. The term “timer” may refer to a clock and/or a predetermined time scheme.

The light generating system may be part of or may be applied in e.g. office lighting systems, household application systems, shop lighting systems, home lighting systems, accent lighting systems, spot lighting systems, theater lighting systems, fiber-optics application systems, projection systems, self-lit display systems, pixelated display systems, segmented display systems, warning sign systems, medical lighting application systems, indicator sign systems, decorative lighting systems, portable systems, automotive applications, (outdoor) road lighting systems, urban lighting systems, green house lighting systems, horticulture lighting, digital projection, or LCD backlighting. The light generating system (or luminaire) may be part of or may be applied in e.g. optical communication systems or disinfection systems.

In yet a further aspect, the invention also provides a lamp or a luminaire comprising the light generating system as defined herein. The luminaire may further comprise a housing, optical elements, louvres, etc. etc. . . . The lamp or luminaire may further comprise a housing enclosing the light generating system. The lamp or luminaire may comprise a light window in the housing or a housing opening, through which the system light may escape from the housing. In yet a further aspect, the invention also provides a projection device comprising the light generating system as defined herein. Especially, a projection device or “projector” or “image projector” may be an optical device that projects an image (or moving images) onto a surface, such as e.g. a projection screen. The projection device may include one or more light generating systems such as described herein. Hence, in an aspect the invention also provides a light generating device selected from the group of a lamp, a luminaire, a projector device, a disinfection device, a photochemical reactor, and an optical wireless communication device, comprising the light generating system as defined herein. The light generating device may comprise a housing or a carrier, configured to house or support, one or more elements of the light generating system. For instance, in embodiments the light generating device may comprise a housing or a carrier, configured to house or support one or more of the one or more first light generating devices, the axicon like optical element, the thermally conductive element, and the optional one or more second light generating devices.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1d schematically depict some embodiments and aspects, and

FIG. 2 schematically depict some application embodiments.

The schematic drawings are not necessarily to scale.

### DETAILED DESCRIPTION OF THE EMBODIMENTS

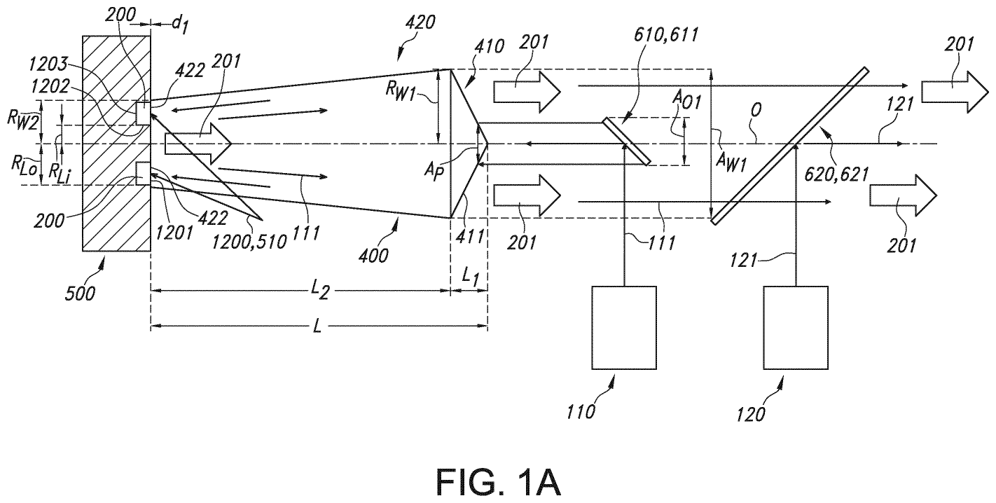

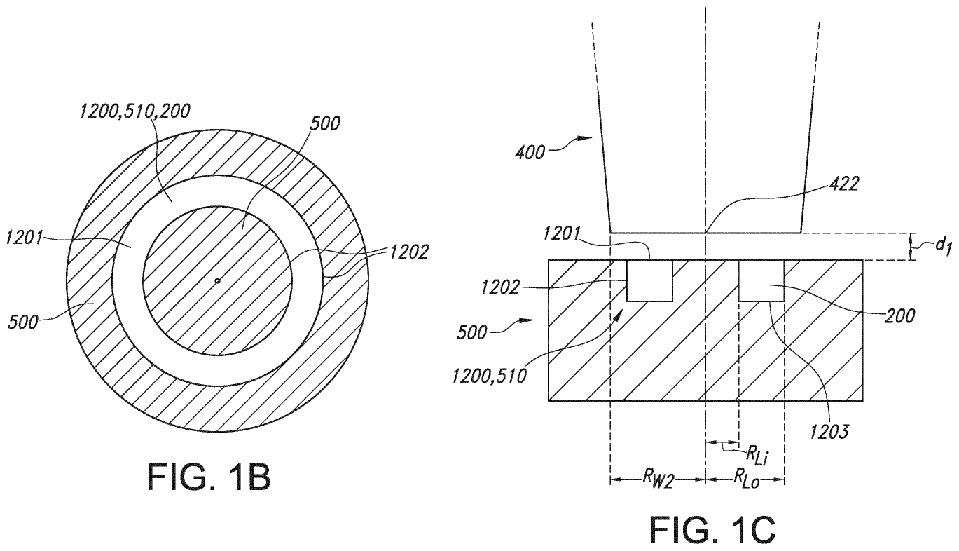

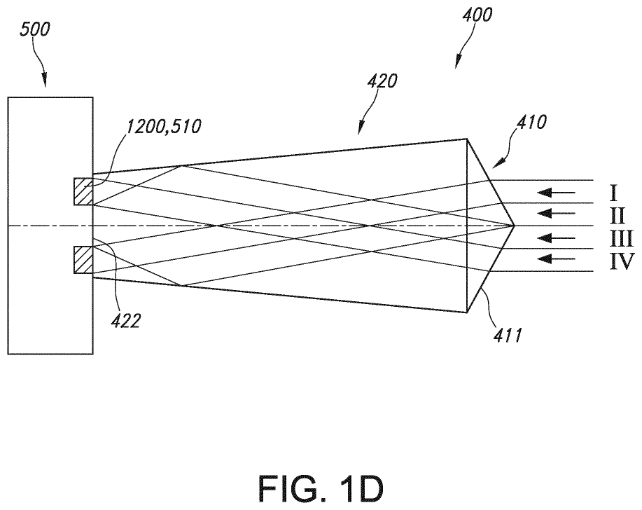

FIG. 1a-1d schematically depict embodiments and aspects in relation to (embodiments of) a light generating system 1000 comprising a first light generating device 110, a luminescent body 1200, a thermally conductive element 500, and an axicon-like optical element 400.

The first light generating device 110 may be configured to generate first device light 111. The first light generating device 110 may comprise one or more of a superluminescent diode and a solid state laser.

The luminescent body 1200 may comprise a luminescent material 200 configured to convert at least part of the first device light 111 into luminescent material light 201. The luminescent body 1200 may have an annular shape (see also FIG. 1b).

The thermally conductive element 500 may optionally comprise an annular slit 510 hosting at least part of the luminescent body 1200. The thermally conductive element 500 may be reflective for one or more of the first device light 111 and the luminescent material light 201.

The axicon-like optical element 400 may comprise a first part 410 and a second part 420, and has an optical element length L. The first part 410 may have a conical shape, a first length L1, and may comprise a first end window 411. The second part 420 may have a cylindrical shape, a second length L2, and may comprise a second end window 422. In embodiments, 0.7≤L2/L<1.

The axicon-like optical element 400 may be configured to: (a) receive at least part of the first device light 111 via the first window part 410 and provide an annular beam of first device light 111 via the second window part 420 to the luminescent body 1200, and (b) collect at least part of the luminescent material light 201 via the second window part 420 and provide a beam of luminescent material light 201 via the first window part 410.

In embodiments, the second part 420 may taper over at least part of the second length L2 in a direction from the first part 410 to the second end window 422.

The first end window 411 may have a largest first window radius (Rw1) and the second end window 422 has a second window radius (R_{W2}). In embodiments, 0.5≤RW2/RW1≤0.98. Further, in embodiments 0.8≤L2/L<1.

The light generating system 1000 may be configured such that part of the first device light 111 escaping from the second end window 422 may be reflected at one or more of the thermally conductive element 500 and the luminescent body 1200, enters after reflection the axicon-like optical element 400 via the second end window 422, and escapes, together with at least part of the luminescent material light 201, via the first end window 411 from the axicon-like optical element 400, to provide a beam of light comprising first device light 111 and the luminescent material light 201.

The first end window 411 may have a largest first window circular cross-section A_{w1}. The optional first optics 610 may comprise a reflector 611, such as in specific embodiments a dichroic mirror 612. The optional first optics may have an optics cross-section Aₒ₁, defined parallel to the pump beam circular cross-section Aₚ and the largest first window circular cross-section A_{w1}. In embodiments, Aₒ₁/A_{w1}≤0.5.

In embodiments, the first light generating device 110 and optional first optics 610 may be configured to provide a beam of first device light 111 at the first end window 411 having a pump beam circular cross-section Aₚ. Further, the first end window 411 may have a largest first window circular cross-section A_{w1}. In embodiments, Aₚ/A_{w1}≤0.8.

In specific embodiments, a first distance d1 between the luminescent body 1200 and the second end window 422 may be selected from the range of 0-0.1*L. In FIG. 1a, d1 is essentially zero, whereas in FIG. 1c, an embodiment is schematically depicted wherein d1 is not zero.

In specific embodiments, and referring to e.g. FIGS. 1a and 1c, the luminescent body 1200 and the second end window 422 may be configured in optical contact.

Referring to FIGS. 1a-1d, the luminescent body 1200 may have an outer luminescent body radius rₗₒ and a luminescent body inner radius rₗᵢ. The second end window 422 may have a second window radius R_{W2}. Especially, Rₗₒ>rₗᵢ. Further, in specific embodiments 0.85≤rₗₒ/R_{W2}<1.

The luminescent body 1200 may have a first face 1201 directed to the second end window 422, side faces 1202, and a bottom face 1203 configured farthest away from the second end window 422. In specific embodiments, the bottom face 1203, and optionally also the side faces 1202, may be configured in thermal contact with the thermally conductive element 500.

In embodiments, the thermally conductive element 500 may be selected from the group comprising a heatsink, a heat spreader, and a two-phase cooling device.

In embodiments, the luminescent material 200 may comprise a luminescent material of the type A₃B₅O₁₂:Ce, wherein A may comprise one or more of Y, La, Gd, Tb and Lu, and wherein B may comprise one or more of Al, Ga, In and Sc. In specific embodiments, the luminescent body 1200 may comprise a ceramic body.

In embodiments, the first light generating device 110 may comprise a blue (laser) light emitting diode laser.

In embodiments, the light generating system 1000 may further comprising a beam combiner 620 and a second light generating device 120. The second light generating device 120 may be configured to generate second device light 121, having a second spectral power distribution different from a first spectral power distribution of the first device light 111, or the spectral power distributions may essentially be the same. In specific embodiments, the second spectral power distribution different from a first spectral power distribution of the first device light 111, and especially, the second device light 121 has intensity in the orange-red wavelength range. The beam combiner 620 may be configured to combine the first luminescent material light 201 downstream of the first end window 411 and the second device light 121 (and optionally the first device light 111). The light generating system 1000 may be configured to generate system light 1001 comprising at least part of the combined first luminescent material light 201, the first device light 111, and second device light 121.

In an operational mode of the light generating system 1000, the system light 1001 may be white light.

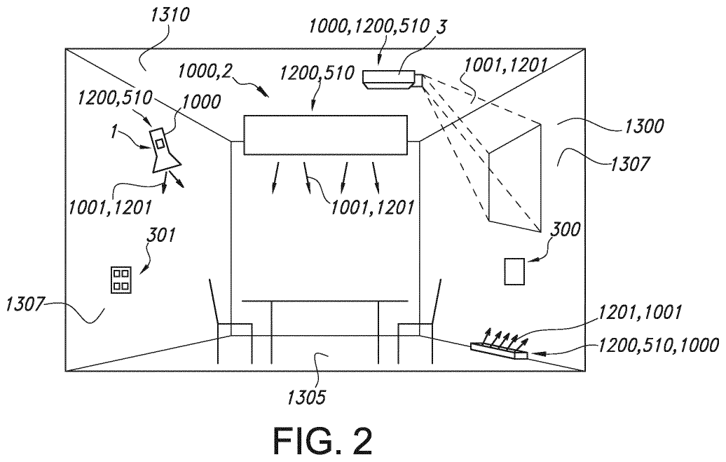

FIG. 2 schematically depicts an embodiment of a luminaire 2 comprising the light generating system 1000 as described above. Reference 301 indicates a user interface which may be functionally coupled with the control system 300 comprised by or functionally coupled to the light generating system 1000. FIG. 3 also schematically depicts an embodiment of lamp 1 comprising the light generating system 1000. Reference 3 indicates a projector device or projector system, which may be used to project images, such as at a wall, which may also comprise the light generating system 1000. Hence, FIG. 3 schematically depicts embodiments of a lighting device 1200 selected from the group of a lamp 1, a luminaire 2, a projector device 3, a disinfection device, a photochemical reactor, and an optical wireless communication device, comprising the light generating system 1000 as described herein. In embodiments, such lighting device may be a lamp 1, a luminaire 2, a projector device 3, a disinfection device, or an optical wireless communication device. Lighting device light escaping from the lighting device 1200 is indicated with reference 1201. Lighting device light 1201 may essentially consist of system light 1001, and may in specific embodiments thus be system light 1001. Reference 1300 refers to a space, such as a room. Reference 1305 refers to a floor and reference 1310 to a ceiling; reference 1307 refers to a wall.

In embodiments, in a configuration a phosphor disk is attached to a reflective heat sink and heat is removed through the back face and side faces of the phosphor tile. When such a disk is irradiated by the pump laser, a hot spot develops in the center of the disk which limits the achievable power densities.

Especially, it is herein proposed to use an annular shape phosphor to increase the contact area with the heat sink and thus prevent overheating of the phosphor by cooling it not only from top and bottom surfaces but also from sides. The annular phosphor can be irradiated with an annular pump beam created using an axicon optical element. The same axicon is then used for collecting the emitted light.

In simulations performed, there is a small air gap between the axicon and the phosphor. However it is also possible to place the axicon directly on top of the phosphor surface for extra cooling and reduce reflections.

The axicon may consist of a tapered cylindrical rod with a conical top surface. With specific values of the cone top angle a hollow, ring-shaped pump beam can effectively irradiate the annular phosphor. It is herein suggested using the same axicon optical element for collecting the converted light, plus the unconverted blue pump light. It is expected that the tapered shape provides a mixing and “pre-collimation” of the converted phosphor light and reflected blue light.

Especially, the blue pump light is coupled in to the axicon using a small dichroic mirror, though a small reflector may also be applied. Part of the blue pump light may be reflected from the phosphor and from the part of the heat sink that may not be covered by the phosphor. The blue reflection of the heat sink can be designed to result in a good white balance of the total output.

Referring to FIG. 1d, it can be seen that light can efficiently be directed onto to the phosphor.

Based on the modelling, it can be concluded that the axicon which projects annular blue light pattern onto the phosphor collects light emitted by the axicon phosphor and makes a circular beam.

In the table 1 below, some of the results for various axicon-phosphor combinations are presented. It can be seen that as expected with increasing phosphor area FWHM also increases:

| Axicon | Axicon | Axicon | Axicon | Phosphor | Phosphor | Phosphor | | | |
|---|---|---|---|---|---|---|---|---|---|
| L | Rw1 | Rw2 | L2 | RLo | RLo − RLi | [Ce:] | CCT | Output | |
| (mm) | (mm) | (mm) | (mm) | (mm) | (mm) | [%] | (K) | [W] | FWHM |
| 5 | 1 | 0.6 | 4.45 | 0.55 | 0.25 | 4 | 7970 | 0.7 | 63° |
| 5 | 1 | 0.8 | 4.3 | 0.75 | 0.3 | 4 | 5740 | 0.62 | 81° |
| 5 | 1 | 0.8 | 4.3 | 0.75 | 0.4 | 2 | 4830 | 0.61 | 81° |
| 5 | 1 | 0.8 | 4.3 | 0.75 | 0.4 | 1 | 5050 | 0.61 | 82° |
| 5 | 1 | 1 | 4.1 | 0.95 | 0.4 | 0.2 | 5270 | 0.58 | 91° |
| 5 | 1 | 0.6 | 4.45 | 0.55 | 0.3 | 4 | 6000 | 0.68 | 63° |

The term “plurality” refers to two or more.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage. In yet a further aspect, the invention (thus) provides a software product, which, when running on a computer is capable of bringing about (one or more embodiments of) the method as described herein.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A light generating system comprising a first light generating device, a luminescent body, a thermally conductive element, and an axicon-like optical element; wherein:
the first light generating device is configured to generate first device light; the first light generating device comprises one or more of a super luminescent diode and a solid state laser;
the luminescent body comprises a luminescent material configured to convert at least part of the first device light into luminescent material light; the luminescent body has an annular shape;
the thermally conductive element (a) is configured in thermal contact with at least part of the luminescent body, and (b) is reflective for one or more of the first device light and the luminescent material light;
the axicon-like optical element comprises a first part and a second part, and has an optical element length (L); the first part has a conical shape, a first length (L1), and comprises a first end window; the second part has a cylindrical shape, a second length, (L2) and comprises a second end window; wherein 0.7≤L2/L<1; and
the axicon-like optical element is configured to: (a) receive at least part of the first device light via the first part and provide an annular beam of first device light via the second part to the luminescent body, and (b) collect at least part of the luminescent material light via the second part and provide a beam of luminescent material light via the first part.

2. The light generating system according to claim 1, wherein the second part tapers over at least part of the second length (L2) in a direction from the first part to the second end window.

3. The light generating system according to claim 2, wherein the first end window has a largest first window radius (Rw1), wherein the second end window has a second window radius (R_{W2}), wherein 0.5≤RW2/RW1≤0.98; and wherein 0.8≤L2/L<1.

4. The light generating system according to claim 1, wherein the light generating system is configured such that part of the first device light escaping from the second end window is reflected at one or more of the thermally conductive element and the luminescent body, enters after reflection the axicon-like optical element via the second end window, and escapes, together with at least part of the luminescent material light, via the first end window from the axicon-like optical element, to provide a beam of light comprising first device light and the luminescent material light.

5. The light generating system according to claim 1, further comprising first optics; wherein the first end window has a largest first window circular cross-section (A_{w1}); wherein the first optics (610) comprises a dichroic mirror(612), having an optics cross-section(Aₒ₁), defined parallel to the largest first window circular cross-section(A_{w1}), wherein Aₒ₁/A_{w1}≤0.5.

6. The light generating system according to claim 5, wherein the first light generating device and first optics are configured to provide a beam of first device light at the first end window having a pump beam circular cross-section(Aₚ); wherein the first end window has a largest first window circular cross-section (A_{w1}) as defined in claim 5, wherein Aₚ/A_{w1}≤0.8.

7. The light generating system according to claim 1, wherein a first distance (d1) between the luminescent body and the second end window is selected from the range of 0-0.1*L.

8. The light generating system according to claim 1, wherein the luminescent body and the second end window are configured in optical contact, wherein the luminescent body has an outer luminescent body radius (R_{Lo}) and a luminescent body inner radius(R_{Li}), wherein the second end window (422) has a second window radius(R_{W2}), wherein R_{Lo}>R_{Li} and wherein 0.85≤R_{Lo}/R_{W2}<1.

9. The light generating system according to claim 1, wherein the thermally conductive element (a) comprises an annular slit hosting at least part of the luminescent body, wherein the luminescent body has a first face directed to the second end window, side faces, and a bottom face configured farthest away from the second end window, wherein the side faces, and the bottom face are configured in thermal contact with the thermally conductive element.

10. The light generating system according to claim 1, wherein the thermally conductive element is selected from the group comprising a heatsink, a heat spreader, and a two-phase cooling device.

11. The light generating system according to claim 1, wherein the luminescent material comprises a luminescent material of the type A₃B₅O₁₂:Ce, wherein A comprises one or more of Y, La, Gd, Tb and Lu, and wherein B comprises one or more of Al, Ga, In and Sc.

12. The light generating system according to claim 1, wherein the luminescent body comprises a ceramic body.

13. The light generating system according to claim 1, wherein the first light generating device comprises a blue light emitting diode laser.

14. The light generating system according to claim 1, further comprising a beam combiner and a second light generating device, wherein the second light generating device is configured to generate second device light, having a second spectral power distribution different from a first spectral power distribution of the first device light; wherein the second device light has intensity in the orange-red wavelength range; wherein the beam combiner is configured to combine the first luminescent material light downstream of the first end window and the second device light; wherein the light generating system is configured to generate system light comprising at least part of the combined first luminescent material light, the first device light, and second device light; wherein in an operational mode of the light generating system, the system light is white light.

15. A lighting device selected from the group of a lamp(1), a luminaire(2), a projector device(3), a disinfection device, a photochemical reactor, and an optical wireless communication device, comprising the light generating system according to claim 1.
